(19) United States
(12) Patent Application Publication
(10) Pub. No.: US 2021/0260300 A1
(43) Pub. Date: Aug. 26, 2021

(54) AN INJECTION DEVICE

(71) Applicant: SUN PHARMA ADVANCED RESEARCH COMPANY LIMITED, Andheri (East), Mumbai (IN)

(72) Inventors: Satyashodhan PATIL, Tandalja Vadodara (IN); Ajay KHOPADE, Tandalja Vadodara (IN); Vivek PATEL, Tandalja Vadodara (IN)

(73) Assignee: SUN PHARMA ADVANCED RESEARCH COMPANY LIMITED, Andheri (East), Mumbai (IN)

(21) Appl. No.: 17/059,645

(22) PCT Filed: May 31, 2019
(86) PCT No.: PCT/IB2019/054549

§ 371 Date: Nov. 30, 2020

(30) Foreign Application Priority Data
Jun. 1, 2018 (IN) 201821020556

(51) Int. Cl. A61M 5/315

(52) U.S. Cl. CPC A61M 5/31536

(57) ABSTRACT

The present invention relates to an injection device for subcutaneous or intramuscular injection of a pharmaceutical formulation characterized by a yield value, said device comprising, a longitudinally continuous hollow element forming a reservoir for the pharmaceutical formulation, said longitudinally continuous hollow element having a first cylindrical hollow element having an internal diameter (d1), in continuum with a second tapering element that tapers from the internal diameter (d1) to an opening with a diameter (d2) over a length (l), wherein d1 ranges from 2.6 mm to 3.0 mm, d2 ranges from 0.71 mm to 1.01 mm and l ranges from 0.91 mm to 1.41 mm; a needle adapted to be attached to the opening of said second tapering element; and a plunger rod for insertion in the said cylindrical hollow element having a thumb rest at one end and a stopper at the other end.

### FIELD OF THE INVENTION

The present invention relates to an injection device for subcutaneous or intramuscular injection of a formulation characterized by a yield value.

### BACKGROUND

Parenteral delivery of medicaments is an invasive delivery where the formulation is administered to a subject by means of a hollow needle of a syringe or catheter across skin and membrane barrier. The medicament in a pharmaceutically acceptable formulation is injected via suitable routes such as intravenous, intramuscular, subcutaneous, intradermal, intrathecal, epidural, intracardiac, intra-articular etc. This enables the medicament to be available in the systemic circulation or at the site of action rapidly. The medicament exerts its action over a period that depends on the biological half-life of the drug and thus parenteral delivery of conventional parenteral formulation is unsuitable for chronic conditions where the patient will require frequent administration. One of the means to prolong the action of such chronically administered medicaments is to formulate the drug in a slow release formulation that may be injected subcutaneously or intramuscularly. The slow release formulation is usually placed in vicinity of capillaries such as by injecting it intramuscularly or subcutaneously, wherefrom the medicament is slowly released from the formulation and then diffuses into the capillaries, thereby providing a sustained and longer duration of action of the drug and reducing the frequency of administration. The present inventors found that when the slow release formulation is of such rheology that it begins to flow only at a particular yield value or stress then conventional injection devices are unsuitable. Such devices were found to pose stiff resistance to flow of the formulation from the reservoir into the needle, thus requiring more force to inject and hence, more the pain at the injection site. Also it would require a longer time for delivering the formulation, making it unsuitable for administration of such a formulation. Thus, there is a need for a device that is adapted to inject such formulation without the above mentioned difficulties.

### SUMMARY OF THE INVENTION

The present inventors have found a device for convenient subcutaneous or intramuscular injection of a pharmaceutical formulation which is characterized by a yield value. More particularly, the present invention provides an injection device that conveniently delivers such a pharmaceutical formulation characterized by a yield value with reduced injection force in a reasonable injection time. More particularly the device is capable of conveniently injecting subcutaneously or intramuscularly a non-Newtonian fluid with viscoelastic properties.

### BRIEF DESCRIPTION OF THE DRAWINGS

The various embodiments of present invention are described in detailed description with the accompanying drawings, in which:

FIG. 1 shows an illustration of side view of an exemplary embodiment of the present invention.

FIG. 2 shows an illustration of cross sectional longitudinal view of an exemplary embodiment of the present invention when the reservoir is filled with a pharmaceutical formulation.

FIG. 3 shows an illustration of cross sectional front view of an exemplary embodiment of the present invention when a pharmaceutical formulation has been dispensed from the reservoir.

FIG. 4 shows an illustration of cross sectional front view of two different exemplary embodiments of inner section of the continuous hollow element in connection with the hub of the needle.

FIG. 5 shows an illustration of a cross sectional view of a stopper.

FIG. 6 shows an illustration of an isometric view of a stopper.

### DETAILED DESCRIPTION

The present invention provides a device for subcutaneous or intramuscular injection of a pharmaceutical formulation characterized by a yield value. The present invention provides an injection device that may be used to conveniently deliver such a pharmaceutical formulation, particularly a non-Newtonian liquid with viscoelastic properties. The delivery is achieved with reduced injection force in a reasonable injection time as compared to the conventional injection devices available in the prior art.

The injection device, provided in the present invention is for subcutaneous or intramuscular injection of a pharmaceutical formulation characterized by a yield value that comprises
i. a longitudinally continuous hollow element forming a reservoir for the pharmaceutical formulation, said longitudinally continuous hollow element having a first cylindrical hollow element having an internal diameter (d₁), in continuum with a second tapering element that tapers from the internal diameter (d₁) to an opening with a diameter (d₂) over a length (l) wherein d₁ ranges between 2.6 mm to 3.0 mm, d₂ ranges between 0.71 mm to 1.01 mm and l varies between 0.91 mm to 1.41 mm;
ii. a needle adapted to be attached to the opening of said second tapering element; and
iii. a plunger rod for insertion in the said cylindrical hollow element having a thumb rest at one end and a stopper at the other end.

The phrase, “pharmaceutical formulation with a yield value” means formulations characterised by a yield value, including but not limited to formulations with viscoelastic properties or formulations exhibiting pseudo plastic, dilatant, thixotropic or rheopecty behaviours. The yield value (commonly called “yield point”) is the resistance to initial flow, or represents the stress required to start fluid movement. Yield value can be measured by methods well known in the art, for e.g., using the Brookfield yield value extrapolation method or Oscillatory tests involving amplitude sweeps that are performed at different amplitude keeping the frequency and temperature constant.

Some examples of such “pharmaceutical formulations with a yield value” are described in WO2017/168435, which is incorporated herein by reference. One such example is liraglutide viscoelastic gel composition is as below:

| Sr. | | Quantity |
|---|---|---|
| No. | Ingredients | (% w/w) |
| 1 | Liraglutide | 15 |
| 2 | Arginine | 3.8 |
| 3 | Water for injection | 23.3 |
| 4 | Soy Phosphatidylcholine (Lipoid S 100) | 24.6 |
| 5 | Glycerol Oleates mixture (IMWITOR ® 948) | 24.6 |
| 6 | Ethanol Absolute 99.9% | 5.8 |
| 7 | Propylene Glycol USP, | 2.9 |
| | Total | 100 |

The yield value and flow point of the composition was measured by Anton Paar MCR 302 rheometer using parallel plate fixture with 25 mm diameter at gap of 1 mm. The strain amplitude was varied logarithmically from 0.001 to 100% at constant frequency of 1 Hz (or 10 rad/s) and 25° C. temperature. The yield value was determined to be 1902 Pa and flow point to be 2448 Pa. Such a composition may be delivered using the injection device of the present invention.

The term “viscoelastic properties” refers to a formulation which when measured in Anton Paar MCR 302 rheometer using parallel plate fixture with 25 mm diameter at gap of 1 mm and varying the strain amplitude logarithmically from 0.001 to 100% at constant frequency of 1 Hz (or 10 rad/s) and 25° C. temperature, the formulation demonstrated a yield value between 200 Pa and 3000 Pa; and a flow point from 300 Pa to 3500 Pa. The formulation with “viscoelastic properties” follow non-Newtonian dynamics.

The term “tapering element” refers to decrease in diameter, either linearly or curvilinearly forming a conical, curved or bowl shaped profile with an opening.

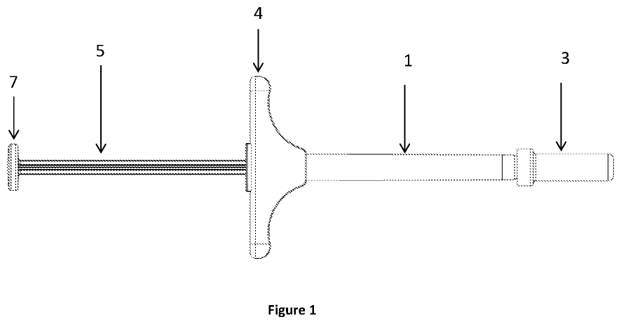

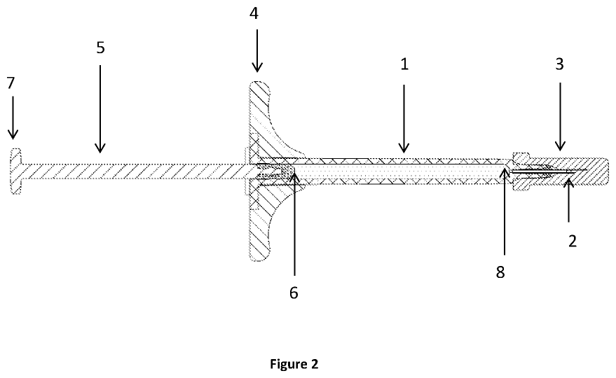

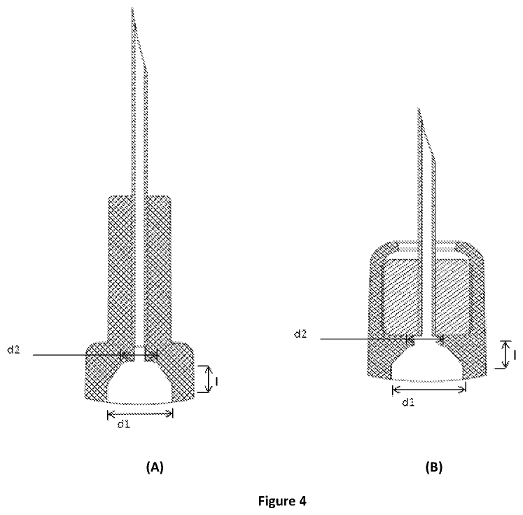

According to the present invention, there is provided a device for delivering a pharmaceutical formulation having a yield value, particularly a non-Newtonian liquid having viscoelastic properties. The device comprises a longitudinally continuous hollow element forming a reservoir, where the reservoir may be filled with the pharmaceutical formulation to be injected. (See FIGS. 1 and 2). The longitudinal hollow element (1) forming the reservoir comprises of two parts in connection with each other, namely, the first cylindrical hollow element and a second tapering element. One end of the cylindrical hollow first element is attached in continuum with the tapering hollow second element. The cylindrical hollow first element has a constant internal diameter (d₁) while the tapering hollow second element tapers from the internal diameter (d₁) to an opening with a diameter (d₂) over a length (l). (See FIG. 4). Whereas, one end of the first cylindrical element is connected to the second tapering element, the other open end is connected to a finger grip.

The injection device also comprises a needle which is adapted to be attached to the tapering hollow second element towards the end of the said element which has an opening with a diameter (d₂). Thus, whereas one opening of the tapering element (with diameter d₁) is connected to the first cylindrical hollow element, the other end is adapted to be attached to a needle. The injection device of the present invention also comprises a plunger rod for insertion in the said cylindrical hollow element having a thumb rest at one end and a stopper at the other end.

Referring now to the drawings, in which reference numerals identify different elements throughout several views, there is shown in FIG. 1 a side view of an exemplary embodiment of the present invention and in FIG. 2, a cross sectional front view of an exemplary embodiment according to the present invention when a pharmaceutical formulation is not dispensed.

There is shown in FIG. 1 and FIG. 2, an exemplary embodiment of a device according to the present invention when a pharmaceutical formulation has not been dispensed, said device comprising a longitudinally continuous hollow element (1) forming a reservoir for holding the formulation to be injected, having a needle (2) with a needle cover (3) removably attached to the said hollow element at one end towards the injection site and finger grip (4) on the other end of the said hollow element. A plunger rod (5) is movably arranged in the continuous hollow element, the said plunger rod has a stopper (6) at the end towards the tapering hollow second element and a thumb rest (7) on the other end.

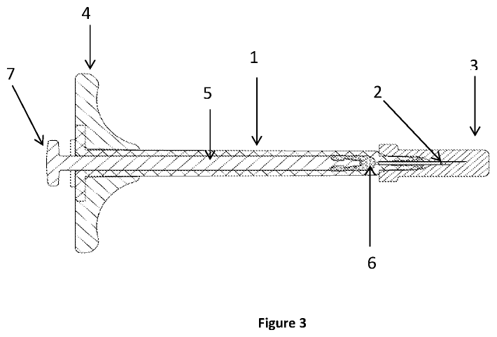

FIG. 3 shows a cross sectional front view of an exemplary embodiment according to the present invention when a pharmaceutical formulation is fully dispensed having the plunger rod (5) forced into the continuous hollow element with said stopper (6) reaching its innermost position.

The plunger is snugly fit into the cylindrical hollow element. When the plunger rod is forced into the said continuous hollow element that forms the reservoir, the stopper on the plunger rod forces the said formulation towards the needle. The plunger rod is forced until the stopper reaches the innermost position of the tapering hollow second element, resulting in complete delivery of the solution. The whole injection device designed as disclosed above facilitates a smooth dispensing of the formulation.

Each component of the device is herein described in details. The longitudinal hollow element that forms a reservoir may be filled with the pharmaceutical formulation. There are two parts of the longitudinal hollow element, a first cylindrical element and a second tapering element; both the parts are in connected to each other at one end. The first cylindrical element has a constant internal and external diameter. The internal diameter d₁ is from 2.6 mm to 3.0 mm. The external diameter ranges from 4.8 mm to 5.6 mm the length of the first cylindrical element ranges from 30 mm to 80 mm.

The material of construction of the continuous hollow element could be glass or plastic. There are different types of plastics used for syringe barrel. Preferably, the said hollow element is formed from a resilient plastic material of medical grade including but not limited to cyclic olefin copolymer (COC) or cyclic olefin copolymer (COP) or Polypropylene (PP). The syringe plunger rod is a rigid rod made of a high strength material including but not limited to steel or an engineering plastic such as polycarbonate, acrylonitrile butadiene styrene (ABS), or Dekin® or polyoxymethylene (acetal) resin of medical grade to withstand pressure of the force to inject high viscous formulation. It could be also made of stainless steel. It is marked/scored to deliver the appropriate volume of the pharmaceutical formulation.

The longitudinal hollow element has smooth internal surface, so that movement of stopper can become uniform and with minimum or negligible friction. Inner surface of syringe barrel can be siliconised to make smooth or frictionless movement of stopper inside it. All of the glass syringes comes in siliconised inner surface. The plunger rod is preferably coated with a low friction material, such as a fluorinated hydrocarbon resin, or a silicone resin, or any other similarly low friction, preferably resilient.

The continuous hollow element, forming the reservoir, is filled with the fluid or pharmaceutical formulation. The finger grip is present at one end of the continuous hollow element that is opposite to the needle attachment end. The finger grip facilitates on holding and stabilizing the device during administration of the formulation to a patient.

The tapering hollow element has an inner section with defined profile which includes, but not limited to conical, reverse dome or bowl shape. The inner section of the end of the continuous hollow element which is near to the needle attachment site is optimised such that the pharmaceutical formulation, particularly a non-Newtonian liquid having viscoelastic properties, can move forward smoothly particularly at the junction of the said hollow element and needle assembly.

The profile of the inner section, where reservoir joins the needle attachment region in the device, is defined by Inner diameter (d₁) of the continuous hollow element, diameter (d₂) of the tapering element where converging profile ends and length (l) between two points where converging profile starts and ends respectively. An optimised profile of the inner section where reservoir joins the needle hub, the first diameter (d₁) is in the range from 2.6 mm to 3.0 mm, the second diameter (d₂) is in the range from 0.71 mm to 1.01 mm and length (l) is in the range from 0.91 mm to 1.41 mm Such defined section having converging ends facilitate the forward movement of the pharmaceutical formulation, particularly a non-Newtonian liquid having viscoelastic properties, with less friction.

More particularly, the profile of the inner section where converging profile starts and ends, the first diameter (d₁) ranges between 2.7 mm and 2.9 mm, the second diameter (d₂) ranges between 0.76 mm and 0.96 mm and length (l) varies between 0.96 mm and 1.36 mm.

The cross sectional front view of an exemplary embodiment of inner contour (8) of the continuous hollow element in connection with the hub of the needle is shown in FIG. 4, identifying the diameter (d₁), diameter (d₂) and length (l), thereby connecting the said hollow element with the needle for smooth flow of the viscoelastic gel or semi-solid formulation.

Inner diameter of the continuous hollow element has been kept as minimum as possible such that the force required to push the stopper forward is minimum. The inner diameter of the continuous hollow element has been optimised based on the plunger rod which is used to push the stopper forward.

The needle has two open ends, one of which remains inside the syringe barrel. Needles are made out of metal and come in different gauges. Needle gauge is defined by its inner bore diameter, outer diameter and wall thickness. Needle shape at the end could be of 3, 4 or 5 bevels.

In one embodiment, the device is provided with a syringe system in which needle is pre-attached to the syringe barrel at front end. In this configuration, the syringe moulding is done with the needle attached to it.

In another embodiment, the device is provided with a syringe system in which needle is pre-attached to the syringe barrel at front end. In this configuration, the needle is attached to the barrel with the help of an adhesive.

In another embodiment, the device is provided with a syringe system in which needle is pre-attached to the syringe barrel at front end. In this configuration, the needle hub will be mechanically fitted with the barrel.

In yet another embodiment, the device is provided with a syringe system in which needles can be attached at the front end of the syringe manually before the injection.

FIG. 4 shows illustrations of two different embodiments of syringe. (A) shows a syringe system in which the needle is pre-attached to the continuous hollow element at the end towards the injection site. In this configuration, syringe moulding is done with the needle attached to it. The needle can also be attached with the help of an adhesive. (B) shows the continuous hollow element in another preferred embodiment, having a needle hub towards the injection site attached to a needle. The needle is attached to the barrel either with the help of an adhesive or by mechanical pressing or fitment.

The figures also illustrate an inner diameter (d₁) of the continuous hollow element, diameter (d₂) of the tapering element towards the end where the profile converges and length (l) between d₁ and d₂.

The needle gauge and length of the needle based on the injection depth, volume to be dispensed and time required to dispense has been optimised. For example, when the needle is 21G then insertion pain would be higher as compared to 23G but time taken to dispense the same amount of medicine would be less. According to the present invention, the needle gauge has been optimised within a range from about 21G to about 23G. Needle length is the length between the inner profile at the end of the continuous hollow element and the tip towards the injection site. Needle length is determined based upon whether required injection is subcutaneous or intramuscular. The needle length has been optimised to range between 7 mm and 20 mm. The needle could be of Stainless Steel of different grades, including but not limited to 304L grade or of 316L grade.

The needle shield is used to cover the needle from accidental pricking as well as to maintain sterility or integrity of the formulation inside and is removed prior to injection. The needle shield used is made of medical grade plastics or bromobutyl rubber or flurobutyl rubber.

A plunger rod has a stopper (6) at the end towards the needle attachment site and a thumb rest (7) on the other end (as seen in FIG. 1 and FIG. 2). The stopper (6) is placed inside the syringe barrel at the end of the reservoir opposite to injection site so that fluid always remains inside the syringe barrel.

The finger grip as well as the plunger rod are designed in such a way that it has enough strength to withstand the high forces and can transfer adequate force on the stopper.

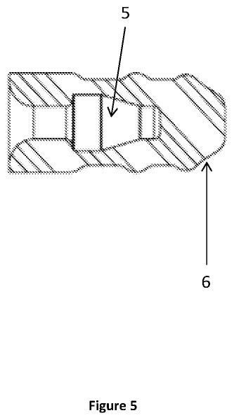

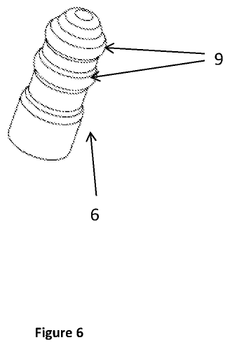

The plunger rod is attached to a stopper. The stopper provided at one end of the plunger rod could be made of rubber or medical grade plastic; more particularly are made of bromobutyl or flurobutyl rubber. Rubber stopper could be of different shapes and length keeping its outer diameter constant. In one embodiment, the rubber stopper is tower shaped with annular rings. Referring now to FIG. 5, it is shown the arrangement of stopper (6) over one end of the plunger rod (5) towards the needle attachment site. FIG. 6 shows the an isometric view of the same stopper (6) displaying tower shaped annular rings (9) that is adapted to snug fit with the continuous hollow element.

To administer a dose of the pharmaceutical formulation, a user has to insert the needle under the skin at injection site of the body and then push the plunger rod forward to dispense the medicine. Adequate amount of force is required to push the plunger rod forward. It becomes uncomfortable and inconvenient to the patient or user if the force required to push the stopper is higher. A principal object of the invention is to reduce the force applied to the plunger to facilitate patient comfort.

While designing a medicament delivery device, there are two factors that are taken into consideration. One is the device component which directly comes in contact with the medicament, while the other is injection, delivery and needle retraction mechanism which can be made either manual or automatic. The needle insertion and drug delivery steps can be made automatic with the help of auto injector device.

One objective is that the device components that come in contact with the medicament do not cause any deteriorating effect on the medicament. Another objective of injection, delivery and needle retraction mechanism is to deliver the medicine at appropriate depth under the skin conveniently and in an appropriate time with appropriate quantity.

The force required to dispense the medicament determines usability of the device and comfort of the patient while administering the medicament. When the force required and time taken to dispense the medicament is very high then patient may feel uncomfortable with the delivery device. It may also become painful for the patient. This is more relevant in case of formulations that have a yield value, particularly non-Neutonian formulation with viscoelastic properties.

For automatic needle insertion and drug delivery of a pharmaceutical formulation characterized by a yield value, delivery mechanism can be based on spring or gear or gas driven technology.

One skilled in the art would appreciate that many other configurations of an injection device utilizing the concepts of the present invention are possible, and such configurations are considered to be encompassed within the appended claims.

1. An injection device for subcutaneous or intramuscular injection of a pharmaceutical formulation characterized by a yield value, said device comprising:
i. a longitudinally continuous hollow element forming a reservoir for the pharmaceutical formulation, said longitudinally continuous hollow element having a first cylindrical hollow element having an internal diameter (d₁), in continuum with a second tapering element that tapers from the internal diameter (d₁) to an opening with a diameter (d₂) over a length (l), wherein d1 ranges from 2.6 mm to 3.0 mm, d₂ ranges from 0.71 mm to 1.01 mm and l ranges from 0.91 mm to 1.41 mm;
ii. a needle adapted to be attached to the opening of said second tapering element; and
iii. a plunger rod for insertion in the said cylindrical hollow element having a thumb rest at one end and a stopper at the other end.

2. An injection device as in claim 1, wherein d1 ranges between 2.7 mm and 2.9 mm, d₂ ranges between 0.76 mm and 0.96 mm and l ranges between 0.96 mm and 1.36 m.

3. An injection device as in claim 1, wherein the pharmaceutical formulation is characterized by a yield value of at least 200 Pa.

4. An injection device as in claim 1, wherein the pharmaceutical formulation is characterized by a yield value of 200 Pa to 3000 Pa.

5. An injection device as in claim 1, wherein the pharmaceutical formulation is a viscoelastic formulation.

6. The injection device as claimed in claim 1, wherein the continuous hollow element forming a reservoir has an internal and an external diameter.

7. The injection device as claimed in claim 5, wherein the continuous hollow element forming a reservoir has an internal diameter from about 0.2 mm to about 5 mm.

8. The injection device as claimed in claim 1, wherein the stopper of the plunger has an external diameter from about 0.2 mm to about 5 mm.

9. The injection device as claimed in claim 1, wherein the stopper of the plunger is made of rubber comprising bromobutyl rubber or flurobutyl rubber.

10. The injection device as claimed in claim 8, wherein the stopper of the plunger is tower shaped with annular rings as depicted in FIG. 6.

11. The injection device as claimed in claim 1, wherein the needle is of a needle length from about 7 mm to about 20 mm.

12. The injection device as claimed in claim 11, wherein the needle is of gauge ranging from 21G to 23G.
